(19) United States
(12) Patent Application Publication
(10) Pub. No.: US 2026/0260715 A1
(43) Pub. Date: Sep. 3, 2026

(54) AN AUTOMATED SYSTEM FOR GENERATING NOVEL MOLECULES

(71) Applicant: TOPIA LIFE SCIENCES LIMITED, London (GB)

(72) Inventor: Kamleshkumar PATEL, London (GB)

(21) Appl. No.: 18/881,928

(22) PCT Filed: Jul. 7, 2023
(86) PCT No.: PCT/GB2023/051800

§ 371 Date: Jan. 7, 2025

(30) Foreign Application Priority Data
Jul. 8, 2022 (GB) 2210089.5

(51) Int. Cl. G16C 20/50; G06N 3/0442; G06N 3/0455; G06N 3/096; G16C 20/70

(52) U.S. Cl. CPC G16C 20/50; G06N 3/0442; G06N 3/0455; G06N 3/096; G16C 20/70

(57) ABSTRACT

There is described as an automated system for generating novel molecules, the system comprising: one or more processors arranged to implement an autoencoder system (300) so as to generate the novel molecules; and an interface for outputting the generated novel molecules, wherein the autoencoder system (300) comprises: i. an encoder (303) configured to: receive input data; and build a latent space (304) based on the input data; and ii. a decoder (305) configured to decode one or more patterns from the latent space (304) so as to generate the novel molecule; wherein said encoder (303) comprises a bidirectional long short-term memory (BiLSTM) model (400) that has been trained independently on a base BiLSTM model.

### FIELD OF THE INVENTION

The present invention relates to an automated system for the generation of novel molecules. Particularly it relates to a system for the generation of novel molecules based on an auto encoder method.

### BACKGROUND OF THE INVENTION

The generation/identification of novel molecules is the first step for finding the novel drugs for any disease. The conventional method of drug discovery takes more than a decade out of which a significant amount of time is spent on identifying the novel molecules. It takes approximately 1 to 2 years to identify the novel molecules for a particular biological target which can be used as a drug. The molecules can either be searched from the chemical space, which is in the order of 10⁶⁰, or a novel molecule can be generated using the inherent knowledge from the known chemical space. Finding the right molecules from such a large space requires domain knowledge, high computation power and time. On the other hand, repurposing the approved drug for a new disease is risky and may come with side effects.

US20170161635A1 relates to generative models. The generative models may be trained using machine learning approaches, with training sets comprising chemical compounds and biological or chemical information that relates to the chemical compounds. Deep learning architectures may be used. In various embodiments, the generative models are used to generate chemical compounds that have desired characteristics, e.g. activity against a selected target. The generative models may be used to generate chemical compounds that satisfy multiple requirements. The disadvantage of the US20170161635A1 is that it works on the chemical compound fingerprints and associated labels for generating drug-like molecules. Further, the system disclosed in it uses probabilistic or variational autoencoders for the generation of novel molecules. Furthermore, the structural evaluation and validation are done using a ranking module and also ranks the generated molecules in various sets based on drug-likeness score.

WO2021180246A1 discloses a drug molecule generation method and apparatus, a terminal device, and a storage medium, which are applicable to the field of digital health. Said method comprises: determining an initial target function value according to graph structure data and SMILES (Simplified Molecular Input Line Entry System) data of a drug molecule; updating atoms in the drug molecule to generate a new drug molecule, and determining a target function value corresponding to the new drug molecule; according to an initial temperature value, the initial target function value, and the target function value, determining whether to accept the new drug molecule; if it is determined to accept the new drug molecule, decreasing the initial temperature value and updating the new drug molecule, and using same as the initial temperature value and the new drug molecule for a next determination of whether to accept new drug molecule, until the initial temperature value in kth iteration is less than a preset temperature threshold, determining a target drug molecule from accepted new drug molecules, outputting the target drug molecule, and displaying same on a terminal The method can be used to improve the accuracy of the design of new drug molecules, so that the reliability of the generated drug molecules is high, further reducing the cost of verification of new drug molecules. In the above-mentioned patent, there are many disadvantages which are described herewith. The input data in the said patent is restricted to FDA-approved drugs only. Hence, there are chances that the generated molecules are present in some other database. Also, said patent is working on a molecular language model along with initial hyperparameters which is purely a language base technique. Furthermore, said patent uses multiple predictive models for various tasks like descriptor generation, classifying the output, generative model etc., which are prone to multiple errors.

WO2022043690A1 discloses a computer-implemented method and system for small molecule drug discovery. In a small molecule drug discovery method, a transition state for a specific enzyme is modelled using quantum mechanics and molecular dynamics-based simulation of the enzyme and substrate reaction; data defining the transition state (a ‘quantum pharmacophore’) is fed to a machine learning engine configured to generate transition state analogues, such as enzyme inhibitors. The disadvantages of the said patents are that it uses structural drug discovery, which is restricted to the extent of chemical space. Also, the system of said patent utilizes the ab initio method, which is computationally expensive and slow. Furthermore, the primary technique to generate the novel molecule in the said patent is quantum mechanics based on the pharmacophore, which involves transition state information which leads to a limited variation in the output, whereas the analogous generated would have a similar basic scaffold.

The problems associated with available drug molecular development systems and methods are addressed here. The main disadvantages of the prior technologies are that it took more than 1-2 months to generate novel molecules. The inventors of the present invention have surprisingly found the solution to all the above-mentioned problems by developing an automated system for the generation of novel molecules as described herein. With the help of the presented system, the time for generating novel molecules can be reduced to a maximum of 2 weeks, ultimately speeding up the rest of the drug discovery process. Instead of finding the molecule from the chemical space and checking whether it could work for the identified biological target, the present invention generates novel molecules with two underlying models, one which learns the patterns of SMILES and another which takes SMILES of approved drugs of the target and generate molecules similar to that target. This will reduce the computation power, dependency on the domain knowledge, and time.

### OBJECT OF THE INVENTION

The principal object of the present invention is to overcome all the mentioned and existing drawbacks of the prior arts by providing a computer-implemented system-based computational model for the generation of novel molecules.

Another object of the present invention is to provide an automated system for the generation of novel molecules using the auto-encoder model.

Another object of the present invention is to provide an automated system for the generation of novel molecules using the SMILES database for accurate results.

Another object of the present invention is to provide an automated system for generating novel molecules, which is fast and accurate compared to conventional technology.

Another object of the present invention is to provide an automated system for the generation of novel molecules, which is a target-based computational model instead of a property-based model.

Another object of the present invention is to provide a model which generates novel molecules using the concept of transfer learning which overcomes the problem faced by the model due to sparse data.

Another object of the present invention is to provide a model that can learn the underlying pattern of the SMILES data, which provides a heterogeneous output that is not restricted to any particular scaffold.

Another object of the present invention is to provide a lightweight and easily trainable model.

Another object of the present invention is to use the generalized data to pre-train the model and use the existing data to overcome the problem of target-specific data scarcity.

### SUMMARY OF THE INVENTION

This summary is provided to introduce a selection of concepts in a simplified form that is further disclosed in the detailed description of the invention. This summary is not intended to identify key or essential inventive concepts of the claimed subject matter, nor is it intended to determine the scope of the claimed subject matter.

The present invention is all about an automated system for the generation of novel molecules.

The main aspect of the present invention is to provide automation for the generation of the novel molecules, wherein said system comprises: one or more processors; a memory to store said input-output data; and an autoencoder system having an encoder configured to make input data compatible with said system and create a latent space; a decoder configured to decode said latent patterns and to generate required novel molecules; said decoder having two dense layers to decode said latent patterns; characterized in that, said encoder having bidirectional long short term memory (BiLSTM) model being capable of learning the sequence of data from left to right and vice-versa; said computational model having two BiLSTM models, one as a base BiLSTM model and other as a derived BiLSTM model; said base BiLSTM is trained of the non-specific data to interpret the SMILES data and the model transfers knowledge of interpreting the SMILES data to derived model, said derived BiLSTM model trained on target specific SMILES to generate novel molecules for defined target.

Another aspect of the present invention is providing a system for the model in which said input SMILES data is in textual form (csv format).

Another aspect of the present invention is to provide a training method for the computational model for the generation of novel molecules comprising the following steps:
a. inputting SMILES data into the base BiLSTM model for training function;
b. extracting said individual SMILES in a matrix form using one-hot encoding;
c. applying data of step b into a BiLSTM model for learning the characteristics of recognized molecules through the training phase and storing in the memory;
d. loading the target-specific file, in csv format, in the BiLSTM derived model and make said data compatible with the model;
e. executing said target-specific data on the model and storing the data after execution;
f. validating the generated SMILES on the basis of structure using RDKit

Yet another aspect of the present invention is to provide a training method for computational models for the generation of novel molecules in which each SMILE is converted into a matrix of 0's and 1's where 1's represents the presence of a particular atom.

According to an aspect of the present disclosure, there is described an automated system for generating novel molecules, the system comprising: one or more processors arranged to implement an autoencoder system so as to generate the novel molecules; and an interface for outputting the generated novel molecules; wherein the autoencoder system comprises: i. an encoder configured to receive input data; and create a latent space based on the input data; and ii. a decoder configured to decode one or more patterns from the latent space so as to generate the novel molecule; wherein said encoder comprises a bidirectional long short term memory (BiLSTM) model (e.g. a derived BiLSTM model) that has been trained independently on a base BiLSTM model.

Preferably, the decoder comprises two dense layers to decode patterns from the latent space.

Preferably, the encoder transforms the input data to be compatible with the BiLSTM model.

Preferably, the interface comprises a user interface and/or a communication interface. Preferably the user interface and/or the communication interface are arranged to output novel molecules.

Preferably, the system comprises a memory to store input data for the computational model and/or output data from the computational model, preferably wherein the memory is arranged to store output data files of the BiLSTM model and/or the base BILSTM model.

Preferably, the input data comprises textual input from a simplified molecular input line system (SMILES).

Preferably, the derived BiLSTM model learns from the knowledge gained by the base BILSTM model.

Preferably, the base BiLSTM model uses SMILES data as input for the purpose of training.

Preferably, the BiLSTM model is arranged to generate the novel molecule independence of the training data received from the base BiLSTM model; and input target data.

Preferably, a computational model is arranged to generate the sequence of the novel molecules for the input target data by training the autoencoder (300) system consisting of BiLSTM layers.

According to another aspect of the present disclosure, there is described a method of training a computational model to generate novel molecules, the method comprising: providing input data to a base BiLSTM model, optionally in csv format of SMILES, so as to train the base BiLSTM model; extracting data points from the input data in a matrix form using one-hot encoding; providing the extracted data points to a derived BiLSTM model so as to train the derived BiLSTM model to learn the characteristics of recognized molecules; providing a target file, in csv format of SMILES, to the derived BiLSTM model, wherein providing the target file comprises specific data so as to make said data compatible with output data of the base BiLSTM model using the concept of transfer learning; receiving novel molecules from the derived BiLSTM model, wherein the novel molecules are generated based on the target file; validating the generated novel molecules on the basis of structure using RDKit.

Preferably, the input data comprises molecules in SMILES format.

Preferably, the method comprises outputting the generated novel molecules and/or saving the generated novel molecules in memory.

Preferably, receiving the novel molecules comprises receiving SMILES data.

Preferably, providing input data comprises: collecting an input data set of SMILES and splitting said data into train, test and validation; creating a dictionary for converting characters to integers and vice versa; converting the SMILES data to an integer format using the said dictionary.

Preferably, providing the extracted data points comprises initializing hyperparameters, like learning rate, number of epochs, and number of layers in the architecture of the derived BiLSTM model; executing the derived BiLSTM model multiple times based on the initialized parameters until a pre-set accuracy has been achieved; and storing the derived BiLSTM model.

Preferably, the method comprises generating a latent space from the encoder model by dividing the layers of the said model into the encoder, latent space and decoder.

Preferably, extracting data points from the input data (e.g. SMILES data) comprises converting the input data into a matrix of 0's and 1's where 1's represents the presence of a particular atom in the input sequence.

Preferably, the method uses the concept of post-padding to normalize the size of the input SMILES before converting the molecules in the form of a matrix of 0's and 1's.

Preferably, the method generates an encoder and decoder model using a Recurrent Neural Network (RNN).

Preferably, the method comprises validating the novel molecules, using RDKit.

According to another aspect of the present disclosure, there is described an automated system for generating novel molecules, the system comprising: a computational model, the computational model comprising an autoencoder system comprising: i. an encoder configured to: receive input data and to transform the input data to be compatible with the said computational model; and determine a latent space based on the input data; and ii. a decoder configured to decode one or more patterns from the latent space so as to generate a novel molecule; wherein said encoder comprises a derived BiLSTM model that has been trained independently that of a base BiLSTM model, and receiving novel molecules from the decoder in accordance with the target file.

### BRIEF DESCRIPTION OF THE DRAWINGS

The foregoing summary and the following detailed description of the invention are better understood when read in conjunction with the appended drawings. For the purpose of illustrating the invention, exemplary constructions of the invention are shown in the drawings.

FIGS. 1a and 1b show the detailed process of a computer-implemented system-based computational model for the generation of novel molecules as described in the present invention.

FIG. 2 shows the auto-encoder model as described in the present invention.

FIG. 3 illustrates the BiLSTM model of the present invention as described in the present invention.

### DETAILED DESCRIPTION OF THE INVENTION

Detailed embodiments of the present invention are disclosed herein. However, it is to be understood that the disclosed embodiments are merely exemplary of the invention, which may be embodied in various forms. Therefore, specific functional and structural details disclosed herein are not to be interpreted as limiting but merely as a basis for the claims and as a representative basis for teaching one skilled in the art to variously employ the present invention in virtually any appropriately detailed structure.

Unless defined otherwise, all technical and scientific terms used herein have the same meaning as commonly understood by one of the ordinary skills in the art to which the invention belongs.

The present invention overcomes the aforesaid drawbacks of conventional systems and methods for the generation of novel molecules. The objects, features, and advantages of the present invention will now be described in greater detail. Also, the following description includes various details and is regarded as merely exemplary. Accordingly, those of ordinary skill in the art will recognize that: without departing from the scope and spirit of the present disclosure and its various embodiments, there may be any number of changes and modifications described herein.

It must also be noted that as used herein and in the appended claims, the singular forms “a”, “an,” and “the” include plural references unless the context clearly dictates otherwise. Although any systems and methods similar or equivalent to those described herein can be used in the practice or testing of embodiments of the present invention, systems are now described.

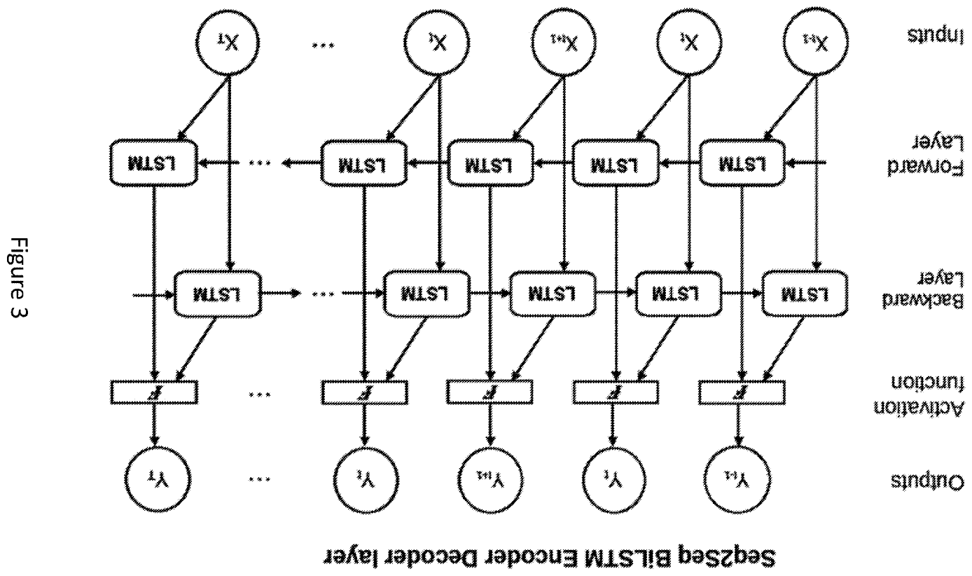

Throughout the specification, the term LSTM is Long Short-Term Memory which is an artificial neural network used in the fields of artificial intelligence and deep learning. Researchers have tried to use the LSTM-based auto-encoder model for novel molecule generation, but the present invention uses the Bidirectional LSTM, also known as BiLSTM. The BiLSTM layers work on the principles of LSTM layers learning in two directions (forward and backward) as shown in FIG. 3 of the present invention.

Throughout the specification, the term SMILES represents the Simplified Molecular Input Line Entry System, a text notation for the topological information based on chemical bonding rules.

Throughout the specification, the term RNN represents the Recurrent Neural Networks. It is a class of Artificial Neural Networks (ANN) in which the connections between nodes form a directed or undirected graph along a temporal sequence. This allows it to exhibit temporal dynamic behaviour.

Throughout the specification, the term Latent Space is an abstract multidimensional space which is capable of encoding a meaningful internal representation of externally observed events. Similar samples in the external world are placed close to each other in the latent space.

Throughout the specification, the term RDKit is the validation software which is used for structural validation of the generated molecules.

The main embodiment of the present invention is to provide an automated system for the generation of novel molecules. An Artificial Intelligence (AI) based model is built using Deep Learning techniques where the model has the capability to learn the molecular data and interpret the patterns. Molecules are represented in a textual format called SMILES. SMILES representation is selected as it is compatible with the RNN model, which is considered as a state-of-the-art model for textual data, which is used as the base of the model.

As per detailed embodiments of the present invention, a system for the generation of novel molecules comprises of one or more processors, a memory to store said input-output data which is in csv format, and an autoencoder system (300) having an encoder (303) configured to make input data compatible with said system and create a latent space (304); a decoder (305) configured to decode the said latent space (304) and generates said novel molecules; said decoder (305) having two dense layers to decode said latent patterns; characterized in that, said encoder (303) having BiLSTM model (400) being capable of learning the sequence of data from left to right and vice-versa, said computational model having two BiLSTM models, one as a base BiLSTM model (100) and other as a derived BiLSTM model (200); said derived BiLSTM model trained by said base BiLSTM model.

As per detailed embodiments of the present invention, said system takes the input data in the form of SMILES representation of molecules which is stored in a csv file. This input SMILES contains data of different molecules. Each SMILES is converted into the fixed length string by embedding the string with additional alphabet at the end to make the sequence of the SMILES even. The model can only understand the numbers, to make the input compatible with the model, one-hot encoding (106) is applied to the data.

As per the detailed embodiment of the present invention, said system comprises two different BiLSTM models: base BiLSTM (100) and derived BiLSTM model (200). To overcome the problem which arises due to the scarcity of data in the model, transfer learning is being used in the present invention. With the use of transfer learning, the base BiLSTM model (100) was trained on the global data having 20,000 SMILES molecules and said BiLSTM base model (100) was reused later for training on target-based SMILES data.

As per the detailed embodiment of the present invention, said derived BiLSTM model (200) has the target file (201) for which the molecule needs to be generated. The derived BiLSTM model (200) also has the trained data file (111), which is received from said base BiLSTM model (100). Said trained data is then utilized with the target file, comprising the target-specific approved molecules in the form of SMILES, to find the novel molecules for the targeted disease. Said computational model is capable of generating the sequence of novel molecules for the specific target by training the autoencoder (300) system along with the BiLSTM models.

As per the detailed embodiment of the present invention, said system has memory to store the output data from the BiLSTM models for further use.

As per the detailed embodiment of the present invention, said training method for the generation of novel molecules comprises the following steps:
a. inputting SMILES data into base BiLSTM model (100) for training function; the input is fed in the form of csv file
b. representing said individual SMILES in a matrix form using the one-hot encoding (106);
c. applying data of step b into a base BiLSTM model (107) for learning the characteristics of molecules through the training phase and storing in the memory (111);
d. loading the target-specific file in csv format (201) in the derived BILSTM model (200) and make the said data compatible (202) with said output data of base BiLSTM model (111);
e. executing said target-specific data on the model (204) and storing the data after execution (205);
f. validating the generated SMILES through step e on the basis of structure using RDKit (208) and saving the generated SMILES (209) in memory.

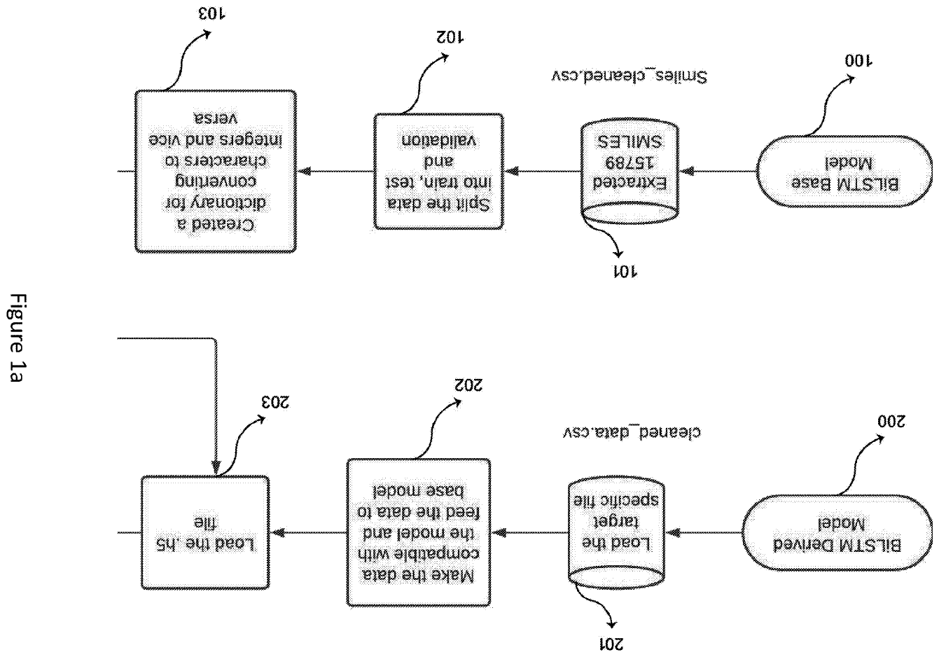

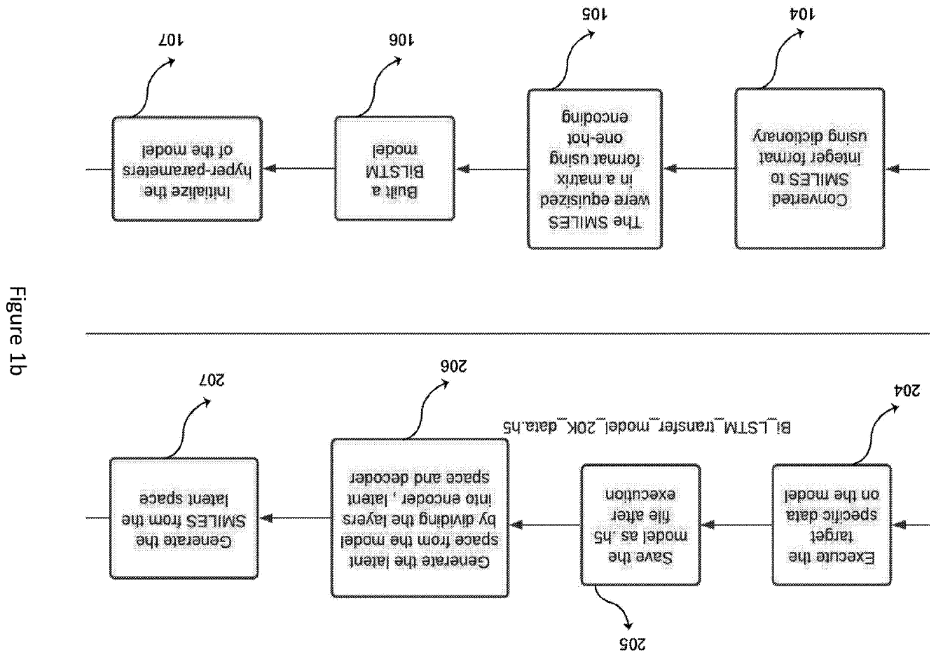

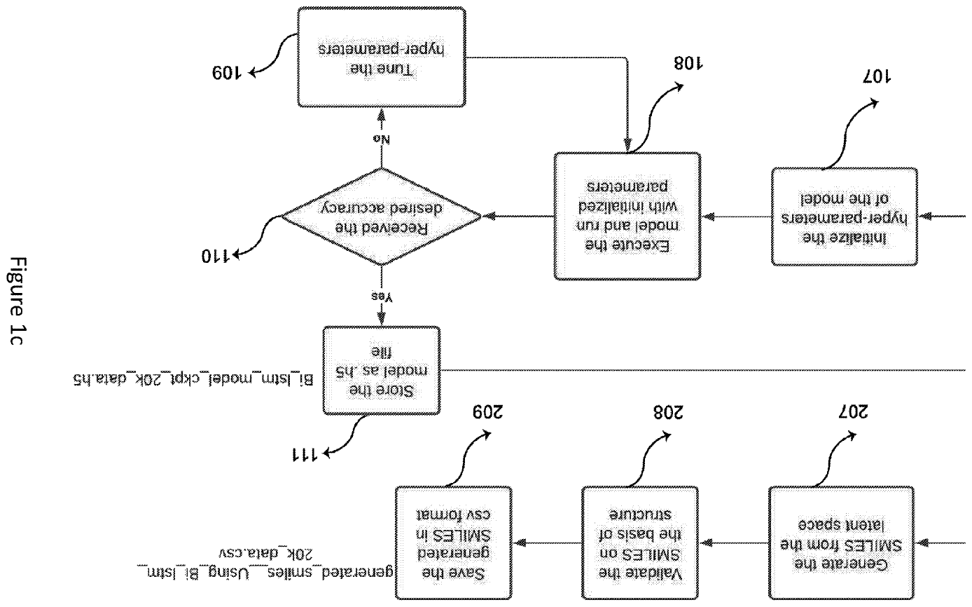

As a detailed embodiment of the present invention, the base BiLSTM model (100) as shown in FIGS. 1a and 1b, takes the input data as SMILES data in the form of .csv file (101). Said base BiLSTM model (100), into the next step (102), split the data into the training, testing and validation form. These trained data need to be converted into integer forms. The model then, in the next step (103), creates a dictionary for converting characters to integer and integer to character. Said SMILES data is then converted into the integer format using said predefined dictionary (104).

As per the detailed embodiment of the present invention, said converted data SMILES were then equalized in a matrix format using the one-hot encoding (105). With this data in the next step, a BiLSTM model was built (106) with hyperparameters of the model initialized as per the requirement. (107). With the conditional parameter (110), the model is executed (109) till the desired accuracy is not achieved. Said trained data is then stored in the memory as the .h5 file, shown in block (111) of FIG. 1b of the present invention.

As per one embodiment of the present invention, said trained data of the base BiLSTM model (100) is then loaded in the derived BiLSTM model (200). The derived BiLSTM model (200) has the property that it takes the input in the similar format as that of the base BiLSTM model (100) but the amount of data is less. With the help of transfer learning, the base BiLSTM model (100) learns the inherent pattern behind the data fed into the model and transfers the learning form base to the derived model.

As per one embodiment of the present invention, the target file (201) which is in the form of csv is loaded in the derived BiLSTM model (200) and said data is further converted into a different form which is compatible with the model (200). Said target-specific file then executes on the model with the trained data of the base BiLSTM model (100), which is shown in block (204) of FIG. 1a. The executed output is then stored in the memory as described in the block (205), as shown in FIG. 1a of the present invention.

As per one embodiment of the present invention, in block (206), the latent space was generated, encoding the data from the encoder. In said latent space, the SMILES are encoded to give the abstract information of the input data. The data from the latent space is given to the decoder to decode it. Decoder has the property that SMILES generated from the latent space are completely novel but, at the same time, have a resemblance to the input data. The generated SMILES are then validated (208) through the RDKit on the basis of structure. The generated SMILES are then saved in the memory in the form of .csv file format.

As per another embodiment of the present invention is that the SMILES being converted into a matrix of 0's and 1's where 1's represent the presence of a particular atom.

As per another embodiment of the present invention, said encoder and decoder model is generated through the Recurrent Neural Network (RNN).

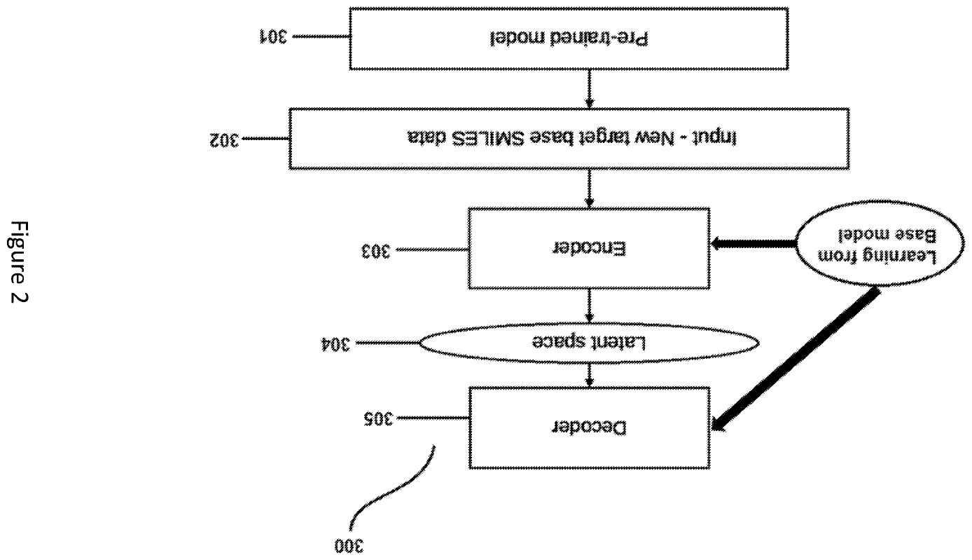

Referring to FIGS. 2 and 3 of the present invention, the core of the computational model is Encoder-Decoder architecture made with BiLSTM. A Bidirectional layer has the ability to learn the sequence in the text from two directions, left to right and right to left. Using a bidirectional layer to parse the string provides a better way to understand the underlying pattern of the SMILES. The Encoder (303) encodes the input and creates a latent space (304) which is then decoded by two dense layers. Said encoder (303) uses a BiLSTM layer, while the decoder has only dense layers.

Without further description, it is believed that one of the ordinary skills in the art can, using the preceding description and illustrative examples, make and utilize the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. An automated system for generating novel molecules, the system comprising:
one or more processors arranged to implement an autoencoder system so as to generate the novel molecules; and an interface for outputting the generated novel molecules; wherein the autoencoder system comprises: an encoder configured to: receive input data; and determine a latent space based on the input data; and
a decoder configured to decode one or more patterns from the latent space so as to generate the novel molecule;
wherein said encoder comprises a bidirectional long short-term memory (BiLSTM) model that has been trained independently of the base BiLSTM model.

2. The system of claim 1, wherein the decoder comprises two dense layers to decode patterns from the latent space.

3. The system, as claimed in claim 1, wherein the encoder transforms the input data to be compatible with the BiLSTM model.

4. The system, as claimed in claim 1, wherein the interface comprises a user interface and/or a communication interface.

5. The system, as claimed in claim 1, comprising a memory to store input data for the computational model and/or output data from the computational model, preferably wherein the memory is arranged to store output data files of the BILSTM model and/or the base BiLSTM model.

6. The system, as claimed in claim 1, wherein the input data comprises textual input from a simplified molecular input line system (SMILES).

7. The system, as claimed in claim 1, wherein the derived BiLSTM model learns from the knowledge transferred by the base BiLSTM model.

8. The system, as claimed in claim 1, wherein the base BiLSTM model uses SMILES data as input to train the base BiLSTM model.

9. The system, as claimed in claim 1, wherein the BiLSTM model is arranged to generate the novel molecule in dependence on: learning received from the base BiLSTM model; and input target data.

10. The system as claimed in claim 1, wherein a computational model is arranged to generate the sequence of the novel molecules for a/the input target data by training the autoencoder system consisting of BiLSTM layers.

11. A method of training a computational model to generate novel molecules, the method comprising:
providing input data to a base BiLSTM model, optionally in csv format of SMILES so as to train the base BiLSTM model; extracting data points from the input data in a matrix form using one-hot encoding; providing the extracted data points to a derived BiLSTM model so as to train the derived BiLSTM model to learn the characteristics of recognized molecules; providing a target file, in csv format of SMILES, to the derived BiLSTM model, wherein providing the target file comprises specific data so as to make said data compatible with output data of the base BiLSTM model using the concept of transfer learning; receiving novel molecules from the derived BiLSTM model, wherein the novel molecules are generated based on the target file; and
validating the generated novel molecules on the basis of structure,
optionally using RDKit.

12. The method, as claimed in claim 11, wherein the input data comprises SMILES data.

13. The method, as claimed in claim 11, comprises outputting the generated novel molecule and/or saving the generated novel molecule in memory.

14. The method, as claimed in claim 11, wherein receiving the novel molecules comprises receiving SMILES data.

15. The method as claimed in claim 11, wherein providing input data comprises:
collecting input data sets of SMILES and splitting said data into train, test and validation; creating a dictionary for converting characters to integers and vice versa; converting the SMILES data to an integer format using the said dictionary.

16. The method as claimed in claim 11, wherein providing the extracted data points comprises:
initializing hyper-parameters, like learning rate, number of epochs, and number of layers in the architecture of the derived BiLSTM model; executing the derived BiLSTM model multiple times based on the initialized parameters until a pre-set accuracy has been achieved; and storing the derived BiLSTM model.

17. The method, as claimed in claim 11, comprises generating a latent space from the encoder model by dividing the layers of the said model into the encoder, latent space and decoder.

18. The method, as claimed in claim 11, wherein extracting data points from the input data (e.g. SMILES data) comprises converting the input data into a matrix of 0's and 1's where 1's represents the presence of a particular atom.

19. The method, as claimed in claim 11, comprises utilizing an encoder and decoder model that uses a Recurrent Neural Network (RNN).

20. The method, as claimed in claim 11, comprises validating the novel molecule using RDKit.

21. An automated system for generating novel molecules, the system comprising:
a computational model, the computational model comprises an autoencoder system comprising:
an encoder configured to receive input data and to transform the input data to be compatible with the said computational model; and build a latent space based on the input data; and
a decoder configured to decode one or more patterns from the latent space so as to generate a novel molecule;
wherein said encoder comprises bidirectional long short-term memory (BiLSTM) model that has been trained independently on a base BILSTM model; and

22. The automated system of claim 21 is being arranged to receive novel molecules from the decoder of the derived BiLSTM model in accordance with a target file.
